(19) United States
(12) Patent Application Publication
(10) Pub. No.: US 2017/0119856 A1
(43) Pub. Date: May 4, 2017

(54) ADMINISTRATION ROUTES OF INSULIN, INSULIN ANALOGS OR DERIVATIVES OF INSULIN

(71) Applicant: SANOFI, Paris (FR)

(72) Inventors: Heinz HAENEL, Frankfurt am Main (DE); Michael SCHABBACH, Frankfurt am Main (DE)

(21) Appl. No.: 15/301,820

(22) PCT Filed: Apr. 23, 2015
(86) PCT No.: PCT/EP2015/058775

§ 371 Date: Oct. 4, 2016

(30) Foreign Application Priority Data
Apr. 25, 2014 (EP) 14305613.3

(51) Int. Cl. A61K 38/28

(52) U.S. Cl. CPC A61K 38/28

(57) ABSTRACT

An insulin, insulin analog or derivative of insulin for use in the treatment of diabetes. The use comprises new administration routes of insulin analogs.

### BACKGROUND OF THE INVENTION

Today, insulin is administered with subcutaneous injection. However, subcutaneous injection is painful and often leads to poor patient compliance. The patients tend to omit their insulin injections because of pain, anxiety and fear associated with the subcutaneous needle.

In addition, the injections of insulin are tightly coupled to the meals of a patient. Every meal must be carefully planned and foregoing insulin injections are required often associated with longer waiting times before the meal can be started.

Thus, one aim in the field of diabetes therapy is a more flexible and convenient delivery of insulin to the patient.

Various minimal invasive delivery methods have been tested. For example inhaled insulin was developed as alternative to subcutaneous insulin injection. This administration route failed patient's and physician's acceptance. Moreover, it still requires subcutaneous injection of basal insulin. Other minimal invasive delivery methods have been developed such as transdermal, oral or buccal methods. These methods are still under investigation for an acceptable bioavailability.

The use of microneedles for intradermal drug delivery has been described in Tuan-Mazlelaa et al. (European Journal of Pharmaceutical Sciences, 2013, 50: 623-37). Microneedles have the advantage that their applications are minimal invasive and more or less painless which makes them attractive for human therapy. Usually, microneedles are made of different materials and geometrical shapes and are micron sized. Typically, they range from lengths as short as 0.025 mm to 2.0 mm.

Microneedles have been tested for the intradermal administration of insulin. It has been shown that the intradermal administration of insulin with stainless steel needles of 1.25 mm, 1.5 mm, and 1.75 mm leads to an improved pharmacokinetic and pharmacodynamic profile compared to subcutaneous administration (Pettis et al., Diabetes Technology & Therapeutics, 2011, 14:435-442; McVey et al, 2012, Journal of Diabetes Science and Technology, 6: 743-754). However, these intradermal injections still require pre-meal injections. Moreover, the long needles may lead to an accidental administration of insulin subcutaneously instead of intradermally with the risk of incorrect dosaging of insulin to the patient.

There is still a need in the art to provide administration routes of insulin, insulin analogs or derivatives of insulin that are more comfortable and safe for the patient.

### DESCRIPTION OF THE INVENTION

The current invention provides an insulin, preferably human insulin or an insulin analog for use in the treatment of diabetes, said use comprising intradermal and post-meal administration of said insulin or insulin analog to a patient.

In addition, the current invention provides an insulin, preferably human insulin or an insulin analog for use in the treatment of diabetes, said use comprising intradermal administration of said insulin or insulin analog to a patient wherein said intradermal administration is with a silicon needle, such as a microneedle. Said administration may occur pre-meal. Said administration may also occur post-meal.

An “insulin analog” as used throughout the application refers to a polypeptide which has a molecular structure which formally can be derived from the structure of a naturally occurring insulin, for example that of human insulin, by deleting and/or exchanging at least one amino acid residue occurring in the naturally occurring insulin and/or adding at least one amino acid residue. The added and/or exchanged amino acid residue can either be codable amino acid residues or other naturally occurring residues or purely synthetic amino acid residues. Examples of analogues of insulin include, but are not limited to, the following:

(i). ‘Insulin aspart’ is created through recombinant DNA technology so that the amino acid B28 in human insulin (i.e. the amino acid no. 28 in the B chain of human insulin), which is proline, is replaced by aspartic acid;
(ii). ‘Insulin lispro’ is created through recombinant DNA technology so that the penultimate lysine and proline residues on the C-terminal end of the B-chain of human insulin are reversed (human insulin: ProB28LysB29; insulin lispro: LysB28ProB29);
(iii). ‘Insulin glulisine’ differs from human insulin in that the amino acid asparagine at position B3 is replaced by lysine and the lysine in position B29 is replaced by glutamic acid;
(iv). “Insulin glargine” differs from human insulin in that the asparagine at position A21 is replaced by glycine and the B chain is extended at the carboxy terminal by two arginines.

Preferably, an insulin analog is a short acting insulin, e.g., selected from insulin glulisine (Apidra®), insulin lispro (Humalog®), and insulin aspart (NovoRapid®).

The present invention further relates to an insulin analog for the uses as described herein.

A “derivative of insulin” as used throughout the application refers to a polypeptide which has a molecular structure which formally can be derived from the structure of a naturally occurring insulin, for example that of human insulin, in which one or more organic substituents (e.g. a fatty acid) is bound to one or more of the amino acids. Optionally, one or more amino acids occurring in the naturally occurring insulin may have been deleted and/or replaced by other amino acids, including non-codeable amino acids, or amino acids, including non-codable, have been added to the naturally occurring insulin. Examples of derivatives of insulin include, but are not limited to, the following:

(i). ‘Insulin detemir’ which differs from human insulin in that the C-terminal threonine in position B30 is removed and a fatty acid residue (myristic acid) is attached to the epsilon-amino function of the lysine in position B29.
(ii). ‘Insulin degludec’ which differs from human insulin in that the last amino acid is deleted from the B-chain and by the addition of a glutamyl link from LysB29 to a hexadecandioic acid.

The present invention further relates to an insulin derivative for the use as described herein.

The inventors of the present invention surprisingly found that the inventive administration routes of insulin analogs have an improved pharmacokinetic and pharmacodynamic profile. Specifically, the cₘₐₓ concentration of the insulin is reached earlier and/or is higher and the decline of insulin levels in the blood occurs more rapidly. The new administration routes further reduce postprandial hypoglycaemias and/or needle fear. Moreover, post-meal administration is more flexible for the patient since it couples administration of insulin to the meal and not vice versa.

“Post-meal” as used herein refers to a time point after the meal. The time point may be immediately after the meal. Preferably the time point is about 1 to about 30 minutes after the meal, about 3 to about 15 minutes after the meal, about 5 to about 10 minutes after the meal, about 1 to about 3 minutes, or about 1 to about 5 minutes after the meal.

“Intradermal administration” as used throughout the application refers to the administration into the dermis of the skin of the patient, preferably the papillary dermis. For examples, intradermal administration is in a depth of about 0.3 mm to about 2.5 mm, preferably of about 0.4 mm to about 2 mm, more preferably of about 0.5 mm to about 1.7 mm, most preferably of about 0.58 to about 0.60 mm, e.g. about 0.58 to about 0.59 mm below the surface of the skin. Intradermal administration has the advantage that it is virtually free of pain.

The administration according to the invention as described herein may occur via injection with any type of needle as long as injection is intradermally.

Preferably injection according to the invention occurs with a microneedle, e.g. a commercially available microneedle, such as a single 1.5 mm stainless steel microneedle as used in the BD Soluvia™ system of Becton Dickinson; a 34-Ga, 1.5 mm microneedle infusion set for connection to infusion pumps (Becton Dickinson), a linear array of etched hollow silicon microneedles as used in the MicronJet needle system (Nano Pass); a circular array of 18 polymer microneedles 500-900 μm in height as used in the hMTSarray (see Pettis et al., 2012, Therapeutic Delivery 3:357-371). The preparation of etched microneedles is described in Yeshurun et al. (U.S. Pat. No. 6,533,949) whose content is also incorporated herein by reference.

The needle or microneedle may be of a variety of materials such as metals, e.g., stainless steel, titanium or nickel-iron, silicon or silicon compounds, glass, ceramic or polymers, e.g., engineering plastics, biodegradable polymers or water-soluble polymers, such as polycarbonate, polylactic-co-glycolic acid and carboxymethyl cellulose, preferably of silicon or silicon compounds.

The needle or microneedle may be of any shape, e.g., cylindrical, pyramidal, rectangular or any other geometrical shape, preferably pyramidally-shaped.

Needles or microneedles as used in the current invention have a length of about 0.2 mm to about 0.5 mm or to about 1.0 mm, or preferably of about 0.4 mm to about 0.9 mm. More preferably the needle or microneedle has a length of about 0.6 mm.

Most preferably, a needle or microneedle used in the current invention is pyramid shaped silica structure with an oblique opening at one of the sides of each pyramid and has a length of 0.6 mm, such as a Micronjet 600™ microneedle (Nanopass Technologies LTD). This leads to a liquid dispersion parallel to the skin layers of the stratum corneum and avoids leakage like in the perpendicular openings from the classical metal microneedles.

Injection with needles or microneedles may occur in any angle relative to the skin as long as the needle is placed intradermally. Preferably, injection with needles or microneedles occurs in a 45° angle relative to the surface of the skin. Injection via a microneedle, particularly a short microneedles, has the advantage that it overcomes the fear of needles that exists with many patients, is minimal invasive, reduces pain and sensations during administration and that it avoids unwanted subcutaneous administration of the insulin analog.

The needle or microneedle of the invention may have a central outlet, for example with a bevel edge opening, or a lateral outlet. Preferably, the needle or microneedle has a lateral outlet. The outlet can adopt any shape such as oval, angled or round shaped, preferably round shaped.

The needles or microneedles of the invention may be used with e.g., patch-like or pump like systems or any standard syringe or pen, the use of those systems of which are known to those skilled in the art (cf. e.g. Escobar-Chavez et al., 2011, J. Clin. Pharmacol. 51:964-977).

The needle or microneedle of the invention may be contained in an array of needles. Preferably, such an array comprises 1 to 50 needles, more preferably 1 to 10, 1 to 5, 1 to 3 or 3 to 8, most preferably 3 needles. The microneedles contained in an array are placed in an equal distance of about 0.2 mm to 1.0 mm, about 0.4 mm to 0.8 mm or about 0.2 mm to about 0.6 mm.

A “patient” as used herein refers to any organism that requires a therapy with insulin, insulin analog or derivative of insulin. Preferably a patient is a patient with a needle phobia, a child, a patient suffering from obesity, a patient starting insulin treatment, a patient with an increased risk for developing postprandial hypoglycemia, and/or a patient using an insulin pump or a patch pump.

The treatment of diabetes as described herein may comprise the treatment of type I and/or type II diabetes. The treatment may also comprise reducing the number postprandial hypoglycemias.

The invention as described herein is particularly useful in the treatment of diabetes with insulin, derivatives of insulin and/or insulin analogs all as described herein.

The injection volume used in the inventive administration route may be lower than the volume used for subcutaneous injection. Particularly, the injection volume is equal or less than 200 μl. Preferably, the injection volume is about 20 μl about 200 μl, about 30 μl—about 170 μl, about 50 μl—about 150 μl, or about 70 μl— about 100 μl.

As used herein, the unit of measurement “U” and/or “international units” and/or “IU” refers to the blood glucose lowering activity of insulin and is defined (according to the World Health Organization, WHO) as follows: 1 U corresponds to the amount of highly purified insulin (as defined by the WHO) which is sufficient to lower the blood glucose level of a rabbit (having a body weight of 2-2.5 Kg) to 50 mg/100 mL within 1 hour and to 40 mg/100 mL within 2 hours.

For human insulin, 100 IU corresponds to approximately 3.5 mg (product information Insuman® Basal). For insulin aspart, 100 U correspond to 3.5 mg (product information NovoRapid®). For insulin lispro, 100 U correspond to 3.5 mg (product information Humalog®). For insulin glulisine, 100 U correspond to 3.49 mg (product information Apidra® cartridges). For insulin determir, 100 U correspond to 14.2 mg (product information Levemir®). For insulin glargin, 100 U correspond to 3.64 mg (product information Lantus®).

The dose of the insulin, insulin analog or derivative of insulin is normally dependent on blood glucose level measured prior to administration and can easily be determined by those skilled in the art. Preferably, the dose is about 0.05 IU/kg, 0.075 IU/kg, 0.1 IU/kg, 0.2 IU/kg, 0.25 IU/kg, 0.3 IU/kg, 0.4 IU/kg, 0.5 IU/kg, 0.7 IU/kg, 1.0 IU/kg, or 2.0 IU/kg, preferably 0.2 IU/kg.

### DESCRIPTION OF FIGURES

FIG. 1

Pharmacokinetic profile of insulin glulisine. The figure shows an earlier tₘₐₓ for intradermal administration.

FIG. 2

Pharmacokinetic profile of insulin lispro. The figure shows an earlier tₘₐₓ, a higher cₘₐₓ and a faster initial elimination slope for intradermal administration.

### EXAMPLES

### Title of the Study:

A randomized, open, single-dose, 4-treatment, 4-period, 4-sequence crossover study of Apidra® and Humalog® intradermal injection (Micronjet 600™ microneedles) compared to subcutaneous injection in healthy subjects using the euglycemic clamp technique (PKD12277).

### Objectives:

The objectives are:
To demonstrate equivalence in overall exposure and activity of Apidra intradermal (ID) injection using Micronjet 600™ compared to subcutaneous (SC) injection.
To demonstrate an increased early exposure and activity of Apidra ID injections using Micronjet 600™ as compared to SC injections.
To demonstrate a decreased late exposure and activity of Apidra ID injections using Micronjet 600™ as compared to SC injections.
To demonstrate equivalence in overall exposure and activity of Humalog ID injection using Micronjet 600™ compared to SC injection.
To demonstrate an increased early exposure and activity of Humalog ID injections using Micronjet 600™ as compared to SC injections.
To demonstrate a decreased late exposure and activity of Humalog ID injections using Micronjet 600™ as compared to SC injections.
To assess the safety and tolerability of Apidra and Humalog with ID administration using Micronjet 600™.

### Methodology:

Open, randomized, crossover (4-treatments, 4-periods, and 4-sequences) in healthy adult male and female subjects.

### Number of: Planned: 28

Randomized: 28
Treated: 28

### Evaluated: Pharmacodynamics: 28

Safety: 28
Pharmacokinetics: 28

### Criteria for Inclusion:

Healthy male and female subjects aged between 18 and 55 years inclusive.

### Study Treatments

### Investigational Medicinal Product (1): Apidra (Insulin Glulisine)

Formulation: 100 U/mL solution for injection
Routes of administration: SC or ID route
Dose regimen: Single dose of 0.2 U/kg, in 2 periods out of 4
Batch number(s): C1023845

### Investigational Medicinal Product (2): Humalog (Insulin Lispro)

Formulation: 100 U/mL solution for injection

Routes of administration: SC or ID route
Dose regimen: Single dose of 0.2 U/kg, in 2 periods out of 4
Batch number(s): C1023804

### Noninvestigational Medicinal Product (1): Glucose (for Euglycemic Clamp)

Formulation: 20% solution for infusion
Route of administration: intravenous (IV) infusion
Dose regimen: as required to maintain a glucose clamp level at 81 mg/dL

### Noninvestigational Medicinal Product (2): Human Soluble Insulin (for Euglycemic Clamp)

Formulation: 100 IU/mL solution for injection
Route of administration: IV infusion
Dose regimen: as required to maintain a glucose clamp level at 81 mg/dL
Noninvestigational Medicinal Product (3): Intramed Heparin Sodium (for Maintenance of catheter permeability)
Formulation: 5000 IU/mL solution
Route of administration: IV infusion
Dose regimen: 10 000 IU in 100 mL 0.9% sodium chloride solution infused at approximately 2 mL/hour

### Noninvestigational Medicinal Product (4): Sodium Chloride (to Keep the Line Patent)

Formulation: 0.9% solution
Route of administration: IV infusion
Dose regimen: infused at approximately 2 mL/hour to keep the catheter patent

### Duration of Treatment:

Between 16 and 48 days from Day-1/Period 1 to end-of-study (EOS), including 4 treatment days (Day 1 of each period) each period comprising an approximate 30 hours in-house, and each separated by a washout period of 3 to 10 days.

### Duration of Observation:

From 2 to 7 weeks maximum (16 to 48 days) excluding the screening period of 3 to 21 days.

### Criteria for Evaluation:

### Pharmacokinetics:

The following pharmacokinetic (PK) parameters were calculated using non-compartmental methods from serum insulin glulisine and insulin lispro concentrations: area under the insulin (INS) concentration time curve from 0 to 10 hours post study drug administration (INS-AUC₀₋₁₀), area under the INS concentration time curve from 0 to 1 hour (INS-AUC₀₋₁₀) and from 4 to 10 hours post study drug administration (INS-AUC₄₋₁₀), maximum insulin concentration (INS-Cₘₐₓ), time to Cₘₐₓ (INS-Tₘₐₓ), times to X % of total INS-AUC₀₋₁₀ (tx %-INS-AUC₀₋₁₀), area under the INS concentration time curve from 1 to 4 hours (INS-AUC₁₋₄) and mean time a molecule resides in the body (MRT).

### Pharmacodynamics:

The following pharmacodynamic (PD) parameters were calculated: area under the body weight standardized glucose infusion rate (GIR) time curve from 0 to 10 hours post study drug administration (GIR-AUC₀₋₁₀); area under the body weight standardized GIR time curve from 0 to 1 hour (GIR-AUC₀₋₁), and from 4 to 10 hours post study drug administration (GIR-AUC₄₋₁₀); times to a X % of total GIR-AUC₀₋₁₀ (tx %-GIR-AUC₀₋₁₀); times to X % of Gift.; maximum smoothed body weight standardized Gift.; time to GIRₘₐₓ (GIR-tₘₐₓ); area under the GIR curve from 0 to 30 minutes, from 0 to 1.5 hours, and from 0 to 2 hours (GIR-AUC_{0-0.5}, GIR-AUC_{0-1.5}, and GIR-AUC₀₋₂, respectively); ratio of GIR-AUC_{0-0.5}/GIR-AUC₀₋₁₀; ratio of GIR-AUC₀₋₁/GIR-AUC₀₋₁₀; ratio of GIR-AUC₀₋₂/GIR-AUC₀₋₁₀; and area under the GIR curve from time t1 to t2 (GIR-AUCₜ₁₋ₜ₂). Times t1 and t2 were defined according to GIR-Tₘₐₓ (t1=mean of GIR-tₘₐₓ+SD and t2=t1+4 hours; i.e. calculated t1=T3 and t2=T7).

Serum C-peptide concentrations were also measured. Glucose clamp performance was evaluated by assessing the blood glucose deviation from the clamp level (81 mg/dL).

### Safety:

Adverse events (AEs) reported by the subject or noted by the Investigator; 12-lead electrocardiogram (ECG); vital signs (systolic blood pressure [SBP], diastolic blood pressure [DBP] and heart rate [HR]); aural temperature; physical examination; clinical laboratory evaluations (hematology, biochemistry, and urinalysis) and injection site reaction assessments (ISR) including injection site pain, erythema, and edema.

### Pharmacodynamics Sampling Times and Bioanalytical Methods:

Venous blood was drawn continuously at a rate of 2 mL/h for the determination of blood glucose every minute. In addition, venous blood samples were collected in 30 minute intervals for concurrent calibration of the Biostator™, which was required for the calibration procedure in order to maintain the glycemic clamp.

Samples for the determination of C-peptide were collected at predose, 1, 2, 3, 4, 5, 6, 7, 8, 9, and 10 hours postdose on Day 1 of each period. Evaluation of C-peptide was conducted using a standard local laboratory assay.

### Pharmacokinetics Sampling Times and Bioanalytical Methods:

Blood samples for the determination of insulin glulisine and insulin lispro concentrations in serum were collected at the following times: predose (−2, −1, −0.5 hours, and 0 hours), 10, 20, 30, 40, and 50 minutes, 1, 1.5, 2, 2.5, 3, 3.5, 4, 4.5, 5, 6, 7, 8, 9, and 10 hours postdose on Day 1 of each period. Serum concentrations of insulin glulisine and insulin lispro were determined using validated bioanalytical methods with lower limits of quantification (LLOQ) of 5 μU/mL for Apidra and Humalog.

### Statistical Methods:

Pharmacokinetics: Pharmacokinetics parameters were summarized by compound and route of administration using descriptive statistics.

For log transformed INS-AUC₀₋₁₀, INS-AUC₀₋₁, INS-AUC₃₋₇, and INS-AUC₄₋₁₀, the ratio of AUCs after ID and SC injections were assessed for each compound using a linear mixed effects model. The estimate and 90% CIs for the route of administration ratio (ID/SC) of geometric means were computed for INS-AUC₀₋₁₀, INS-AUC₀₋₁, INS-AUC₃₋₇, and INS-AUC₄₋₁₀ within the linear mixed effects model framework. Equivalent bioavailability between id and sc route of administrations was concluded if the 90% CI for the route of administration ratio (id/sc) of INS-AUC₀₋₁₀ was entirely contained within 0.80 to 1.25. The INS-tₘₐₓ values were analyzed non-parametrically based on Hodges-Lehmann method for paired treatment comparisons.

Significant higher early exposure for ID versus SC formulations will be concluded if the 90% confidence interval for the formulation ratio (ID/SC) of INS-AUC₀₋₁ is entirely above 1.

Significant lower late exposure for ID versus SC formulations will be concluded if the 90% confidence interval for the formulation ratio (ID/SC) of INS-AUC₄₋₁₀ is entirely below 1. The similar criteria will be applied to the of INS-AUC₃₋₇ ratios.

The analyses were conducted on all patients/periods without any leakage following its administration (referred to as population A). In addition, an analysis was performed on a population including patients/periods with no or only minor leakage (referred to as population AB).

### Pharmacodynamics:

Pharmacodynamics parameters were summarized by compound and route of administration using descriptive statistics. For log transformed GIR-AUC₀₋₁₀, GIR-AUC₀₋₁, GIR-AUC₄₋₁₀ and GIR-AUC₃₋₇, the ratio of AUCs after ID and SC injections were assessed using a linear mixed effects model and the estimate and 90% CI for the difference between treatment means were computed within the linear mixed effects model framework, and then converted to the ratio of geometric means by the antilog transformation. Equivalent bioefficacy (activity) between ID and SC routes of administration was concluded if the 90% CI for the route of administration ratio (ID/SC) of GIR-AUC₀₋₁₀ was entirely contained within the interval 0.80 to 1.25.

GIR-tₘₐₓ was analyzed non-parametrically based on the Hodges-Lehmann method for paired treatment comparisons and 90% CIs for treatment differences were derived.

The individual variability of the blood glucose per clamp was derived as the coefficient of variation (CV %) of blood glucose values between dosing and end of the clamp.

### Safety:

The safety evaluation was based on the review of the individual values (clinically significant abnormalities) and descriptive statistics by compound and route of administration.

For AEs, frequencies of treatment-emergent adverse events (TEAEs) classified by Medical Dictionary for Regulatory Activities (MedDRA) system-organ class and preferred term were tabulated by treatment. All AEs were listed.

For vital signs and ECGs, counts of subjects with abnormalities and PCSAs were summarized by compound and route of administration for each parameter.

Frequencies for signs of local intolerance were analyzed per compound and route of administration.

### SUMMARY

### Population and Injection Assessment:

Twenty eight (28) subjects (20 males and 8 females) were included and completed all 4 treatment periods.

The number of subjects which presented validated injections where no leakage was observed (defined as population A in the protocol) was by treatment period:

24 for intradermal insulin glulisine (ID-GLU) [1 major leakage for Subject No 276001105 and 3 subjects with minor leakages].

28 for subcutaneous insulin glulisine (SC-GLU).

26 for ID insulin lispro (ID-LIS) [1 major leakage for Subject No 276001204 with no bleb formation and 1 subject with minor leakage].

28 for SC insulin lispro (SC-LIS).

The frequency of minor and major leakages following ID administration route of both insulin glulisine and insulin lispro were therefore of 4/56 (7.1%) and 2/56 (3.6%), respectively. There was no IMP leakage following SC administration.

Pharmacokinetic and pharmacodynamic analyses were also extended to subjects who presented minor leakages (population AB) following ID injections in order to broaden the Micronjet 600™ assessment.

### Safety Results:

Seven (7) treatment emergent adverse events (TEAEs) were reported in 4/28 (14.3%) subjects overall.

Within treatment periods, TEAEs were reported in 3/28 subjects for ID-GLU, 1/28 for SC-GLU, 2/28 for ID-LIS and 1/28 for SC-LIS.

Four (4) mild headaches were reported for one subject during each clamp procedure/period.

From tolerability standpoint, some subjects presented mild erythemas following injection (T0h10), which were reported or not as TEAE depending on the injection site reaction (ISR) size and Principal Investigator's assessment.

In total 6/28 subjects presented ISRs following ID-GLU injection (persisting in 3/28 subjects at T2 only for ID-GLU), 1/28 for SC-GLU and 4/28 for ID-LIS., 3 three subjects out of these presented 3 mild injection site erythemas that were recorded as TEAEs following ID injections (2 with ID-GLU, 1 with ID-LIS). No erythema was observed following SC-LIS injections. One (1) subject presented a mild edema following ID-GLU injection (T0h10) that still persisted at T2.

Mean pain scores recorded by visual analog scales (VAS, 0 to 100 mm), were of 27.85 mm (ID-GLU) versus 15.70 (SC-GLU) and of 11.50 mm (ID-GLU) versus 9.88 mm (SC-GLU) during insulin glulisine injections in males and females, respectively. For insulin lispro, they were of 32.70 mm (ID-LIS) versus 6.90 mm (SC-LIS) and of 25.50 mm (ID-LIS) versus 9.75 mm (SC-LIS) in males and females, respectively.

There were no serious adverse events reported during the study.

No clinically relevant abnormalities or PCSAs were recorded for laboratory parameters (hematology, biochemistry and coagulation assessment), vital signs or ECG.

### Pharmacokinetic Results (Population A):

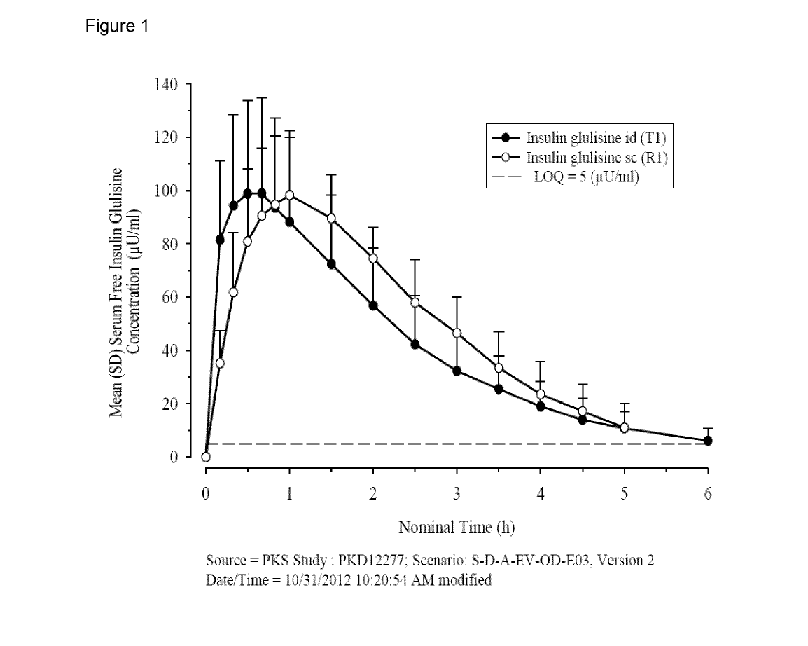

Mean serum insulin glulisine concentration time profiles following ID and SC administration of 0.2 U/kg globally showed a similar shape with nevertheless a shorter median tₘₐₓ following ID administration (0.5 h compared to 1 h). Observed mean Cₘₐₓ values were similar following ID and SC administration. The mean INS-AUC₀₋₁₀ value following ID administration is slightly lower compared to the mean INS-AUC₀₋₁₀ following SC administration (cf. FIG. 1). Early exposure characterized by mean INS-AUC₀₋₁ was higher following ID versus SC administration. Mean INS-AUC₄₋₁₀ and INS-AUC₃₋₇ ratios did not yield significant lower exposure for ID versus SC administration with estimates (and 90% CIs) of 1.26 (0.98; 1.63) and 0.86 (0.68; 1.07), respectively (the significance threshold was met at a upper boundary of 90% CI<1) (cf. FIG. 1).

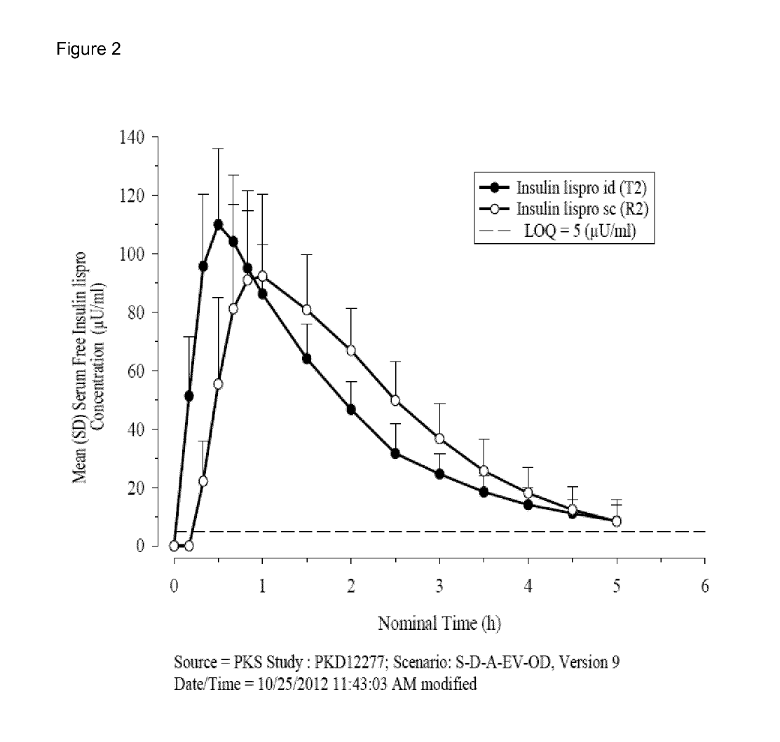

Mean serum insulin lispro concentration time profiles following ID and SC administration of 0.2 U/kg showed a slightly sharper shape for the ID route characterized by a shorter median tₘₐₓ (0.5 h compared to 1 h) and an increased mean Cₘₐₓ value as compared to the SC route (cf. FIG. 2).

Equivalent bioavailability between ID and SC route of administrations was demonstrated since the 90% CI ratio estimate of INS-AUC₀₋₁₀ for ID versus SC route was well inside the defined interval of 0.8-1.25 (90% CI: 0.93-1.02) (cf FIG. 2).

Early exposure characterized by mean INS-AUC₀₋₁ was significantly higher following ID versus SC administration as the 90% CI of the exposure ratio estimate (1.70) was entirely >1 [1.52-1.92].

A lower late exposure was not observed for mean INS-AUC₄₋₁₀ following ID versus SC administration (ratio estimate: 1.11, 90% CI: 0.90-1.36). Conversely, the mean INS-AUC₃₋₇ ratio yield statistically significant lower exposure for ID versus SC administration with an estimate of 0.84 and a 90% CI fully comprised below 1 (0.74-0.95).

Similar conclusions were found between population A and AB.

TABLE 1
PK parameters of insulin glulisine and insulin lispro—population A

| | Mean ± SD (Geometric Mean) [CV %] | | | |
|---|---|---|---|---|
| | Insulin glulisine^{b} | | Insulin lispro^{d} | |
| | ID (T1) | SC (R1) | ID (T2) | SC (R2) |
| N | 24^{c} | 28 | 26^{e} | 28 |
| Cₘₐₓ | 103 ± 36.6 | 101 ± 24.3 | 112 ± 26.2 | 97.4 ± 30.4 |
| (μU/mL) | (90.6) [35.7] | (98.7) [24.0] | (109) [23.5] | (93.5) [31.2] |
| tₘₐₓ^{a} | 0.50 | 1.00 | 0.50 | 1.00 |
| (h) | (0.17-0.83) | (0.50-2.00) | (0.33-0.83) | (0.67-2.00) |
| INS-AUC₀₋₁ | 85.1 ± 30.0 | 68.7 ± 20.1 | 81.7 ± 19.8 | 49.7 ± 19.8 |
| (μU · h/mL) | (75.2) ± [35.2] | (65.9) [29.3] | (79.4) ± [24.2] | (46.4) [39.8] |
| INS-AUC₄₋₁₀ | 37.2 ± 20.7 | 34.8 ± 27.1 | 28.3 ± 17.2 | 27.5 ± 19.8 |
| (μU · h/mL) | (30.6) ± [55.6] | (24.4) [77.7] | (23.0) ± [60.7] | (21.1) [71.9] |
| INS-AUC₃₋₇ | 55.0 ± 26.8 | 64.1 ± 33.3 | 42.7 ± 18.5 | 50.7 ± 24.3 |
| (μU · h/mL) | (46.6) ± [48.6] | (54.8) [52.0] | (38.5) ± [43.2] | (45.2) [48.0] |
| INS-AUC₀₋₁₀ | 264 ± 95.8 | 285 ± 44.5 | 221 ± 40.0 | 235 ± 41.8 |
| (μU · h/mL) | (234) [36.3] | (281) [15.6] | (218) [18.1] | (231) [17.8] |
| INS-AUC | 279 ± 85.6 | 286 ± 46.3 | 234 ± 41.0 | 236 ± 42.5 |
| (μU · h/mL) | (261) [30.7] | (283) [16.2] | (230) [17.5] | (232) [18.0] |

^{a}Median (Min − Max)
^{b}Source = PKS Study: PKD12277; Scenario: S-D-A-EV-OD-E03, Version 2 Date/Time = Oct. 31, 2012 10:20:54 AM modified
^{c}Period profiles of subjects 276001105, 276001109, 276001115, 276001118, 276001119 and 276001204 for ID administration presenting major and minor leakage were excluded
^{d}Source = PKS Study: PKD12277; Scenario: S-D-A-EV-OD, Version 9 Date/Time = Oct. 25, 2012 11:43:03 AM modified
^{e}two patients with minor leakage were excluded

TABLE 2
Treatment effect on INS-AUC₀₋₁₀, INS-AUC₀₋₁, INS-AUC₄₋₁₀,
INS-AUC₃₋₇ and INS-Cₘₐₓ—population A—Point
estimates of treatment ratio with 90% confidence interval

| Treatment ratio | Parameter | Estimate | 90% CI |
|---|---|---|---|
| T1/R1 | INS-AUC₀₋₁₀ | 0.84 | (0.69 to 1.02) |
| T2/R2 | | 0.98 | (0.93 to 1.02) |
| T1/R1 | INS-AUC₀₋₁ | 1.13 | (0.92 to 1.40) |
| T2/R2 | | 1.70 | (1.52 to 1.92) |
| T1/R1 | INS-AUC₄₋₁₀ | 1.26 | (0.98 to 1.63) |
| T2/R2 | | 1.11 | (0.90 to 1.36) |
| T1/R1 | INS-AUC₃₋₇ | 0.86 | (0.68 to 1.07) |
| T2/R2 | | 0.84 | (0.74 to 0.95) |
| T1/R1 | INS-Cₘₐₓ | 0.92 | (0.75 to 1.14) |
| T2/R2 | | 1.13 | (1.04 to 1.24) |

T1: 0.2 U/kg insulin glulisine (Apidra ® intradermal injection (id);
R1: 0.2 U/kg insulin glulisine subcutaneous injection (sc);
T2: 0.2 U/kg insulin lispro (Humalog ®) intradermal injection (id);
R2: 0.2 U/kg insulin lispro subcutaneous injection (sc).
Population A: For intradermal dosing only periods with no leakage are included in the analysis.
PGM = PRODOPS/HMR1964/PKD12277/CSR/REPORT/PGM/pk_equiv_k.sas OUT = REPORT/OUTPUT/pk_ba_a_k_t_2_i.rtf (Nov. 6, 2012-13:50)

### Pharmacodynamic Results:

The clamp quality, assessed by the coefficient of variation of blood glucose (CV %) over the clamp duration (0-10 h), was around 7% for each treatment period and therefore considered adequate (acceptance criteria: <10%).

GIR profiles were globally in agreement with the observed insulins concentration profiles (for both population A and AB, data not shown for population AB).

TABLE 3
Treatment effect on GIRmax, GIR-AUC(0-10 h), GIR-AUC(0-1 h),
GIR-AUC(4-10 h) and GIR-AUC(3-7 h)—[population A]
Point estimates of treatment ratio with 90% confidence intervals

| Treatment ratio | Parameter | Estimate | 90% CI |
|---|---|---|---|
| T1/R1 | GIR-AUC(0-10 h) | 0.87 | (0.71 to 1.05) |
| T2/R2 | | 0.96 | (0.89 to 1.03) |
| T1/R1 | GIR-AUC(0-1 h) | 1.37 | (1.21 to 1.56) |
| T2/R2 | | 1.62 | (1.44 to 1.83) |
| T1/R1 | GIR-AUC(4-10 h) | 0.98 | (0.83 to 1.16) |
| T2/R2 | | 0.85 | (0.71 to 1.02) |
| T1/R1 | GIR-AUC(3-7 h) | 0.75 | (0.54 to 1.03) |
| T2/R2 | | 0.78 | (0.69 to 0.89) |
| T1/R1 | GIRmax | 0.89 | (0.78 to 1.01) |
| T2/R2 | | 0.96 | (0.88 to 1.04) |

T1: 0.2 U/kg insulin glulisine (Apidra ® intradermal injection (id);
R1: 0.2 U/kg insulin glulisine subcutaneous injection (sc);
T2: 0.2 U/kg insulin lispro (Humalog ®) intradermal injection (id);
R2: 0.2 U/kg insulin lispro subcutaneous injection (sc).
GIRmax is based on smoothed GIR profiles.
Population AB: For intradermal dosing only periods with no and minor leakage are included in the analysis.
PGM = PRODOPS/HMR1964/PKD12277/CSR/REPORT/PGM/pd_equiv_d.sas OUT = REPORT/OUTPUT/pd_ba_ab_k_t_2_i.rtf (Nov. 9, 2012-15:10)

Overall glucodynamic activity equivalence quantified by GIR-AUC₀₋₁₀ₕ for ID versus SC ratio is demonstrated for insulin lispro (90% CI of GIR-AUC₀₋₁₀ₕ is [0.90 to 1.03]).

The 90% CIs of GIR-AUC₀₋₁₀ₕ for ID versus SC ratio are entirely above 1 for insulin glulisine and insulin lispro. It demonstrates an increased glucodynamic effect in the early absorption phase of both compounds when administered by ID route versus SC. GiRmax for each compound are virtually equivalent whatever the administration route.

The conclusions derived from pharmacodynamic data statistical analyses that were conducted with population AB are equivalent to those of population A.

1. An insulin or insulin analog for use in the treatment of diabetes, said use comprising intradermal and post-meal administration of said insulin or insulin analog to a patient.

2. An insulin or insulin analog for the use according to claim 1, wherein said administration is via injection with a needle or microneedle.

3. An insulin or insulin analog for use in the treatment of diabetes, said use comprising intradermal administration of said insulin or insulin analog to a patient wherein said intradermal administration is with a silicon needle or silicon microneedle.

4. An insulin or insulin analog for use according to any one of claims 1 to 3, wherein said intradermal administration is in a depth of about 0.3 mm to about 2.5 mm, preferably of about 0.4 mm to about 2 mm, more preferably of about 0.5 mm to about 1.7 mm, most preferably of about 0.58 mm to about 0.59 mm below the surface of the skin.

5. An insulin or insulin analog for the use according to any one of claims 2 to 4, wherein said needle or microneedle has a length of about 0.4 mm to about 0.9 mm, preferably about 0.6 mm.

6. An insulin or insulin analog for the use according to any one of claims 2 to 5, wherein said needle or microneedle has a lateral outlet.

7. An insulin or insulin analog for the use according to any one of claims 2 or 4 to 6 wherein said needle or microneedle is made of silicon.

8. An insulin or insulin analog for the use according to any one of claims 2 to 7 wherein said needle or microneedle is pyramidally-shaped.

9. An insulin or insulin analog for the use according to any one of claims 2 to 8 wherein said needle or microneedle is pyramidally-shaped, has a length of 0.6 mm and is made of silicon.

10. An insulin or insulin analog for the use according to any one of claims 2 to 9, wherein said needle or microneedle is contained in an array of needles or microneedles.

11. An insulin or insulin or insulin analog for the use according to claim 10, wherein said array comprises 1 to 50 needles or microneedles, preferably 1 to 6 needles or microneedles, more preferably 1 to 3 needles or microneedles.

12. An insulin or insulin analog for the use according to any one of the preceding claims, wherein said treatment comprises reducing the number postprandial hypoglycemias.

13. An insulin or insulin analog for the use according to any one of the preceding claims wherein said patient is a patient with a needle phobia, a child, a patient suffering from obesity, a patient starting insulin treatment, a patient with an increased risk for developing postprandial hypoglycemia, and/or a patient using an insulin pump or a patch pump, preferably a patient suffering from obesity.

14. An insulin or insulin analog for the use according to any one of the preceding claims wherein said insulin analog is a short acting insulin analog and/or wherein said insulin is human insulin.

15. An insulin analog for the use according any one of the preceding claims wherein said insulin analog is selected from insulin glulisine, insulin lispro, and insulin aspart.
